(19) United States
(12) Patent Application Publication
(10) Pub. No.: US 2016/0010141 A1
(43) Pub. Date: Jan. 14, 2016

(54) HIGH EFFICIENCY METHODS OF PRODUCING BLOOD GLUCOSE TEST ELEMENTS, AS WELL METHODS OF USING THE SAME

(71) Applicant: ROCHE DIABETES CARE, INC., Indianapolis, IN (US)

(72) Inventors: Claudia Gaessler-Dietsche, Schriesheim (DE); Hans-Peter Haar, Wiesloch (DE); Carina Horn, Biblis (DE)

(73) Assignee: ROCHE DIABETES CARE, INC., Indianapolis, IN (US)

(21) Appl. No.: 14/831,388

(22) Filed: Aug. 20, 2015

Related U.S. Application Data
(63) Continuation of application No. PCT/EP2014/053468, filed on Feb. 21, 2014.

(30) Foreign Application Priority Data
Feb. 22, 2013 (EP) 13156363.7

(51) Int. Cl. C12Q 1/32

(52) U.S. Cl. CPC C12Q 1/32; G01N 2333/904

(57) ABSTRACT

Methods are provided for producing diagnostic test elements, where the diagnostic test elements include a stable analytical chemical reagent of a coenzyme-dependent enzyme and an artificial coenzyme. Diagnostic products including diagnostic test elements having the stable analytical chemical reagent also are provided.

### CROSS-REFERENCE TO RELATED APPLICATIONS

This patent application is a continuation of Int'l Patent Application No. PCT/EP2014/053468 (filed 21 Feb. 2014), which claims priority to and the benefit of EP Patent Application No. 13156363.7 (filed 22 Feb. 2013). Each patent application is incorporated herein by reference as if set forth in its entirety.

### REFERENCE TO SEQUENCE LISTING SUBMITTED ELECTRONICALLY

An official copy of a Sequence Listing is submitted electronically via EFS-Web as an ASCII-formatted Sequence Listing with a file named “26247SequenceListing.txt,” created on 13 Aug. 2015, and having a size of 6 KB. The Sequence Listing is filed concurrently with the Specification, is a part thereof and is incorporated herein by reference as if set forth in its entirety.

### TECHNICAL FIELD

This disclosure relates generally to engineering, chemistry and medical diagnostics, and more particularly, it relates to methods of making diagnostic test elements, to diagnostic test elements including a stable analytical chemical reagent, and to methods of using stable analytical chemical reagent.

### BACKGROUND

Diagnostic test elements are important parts of clinically significant analysis methods. Measuring analytes such as, for example, metabolites or substrates, is of primary importance here, and can be determined directly or indirectly by using an enzyme specific for the analyte. Typically, analytes are converted using an enzyme-coenzyme complex and subsequently quantified using suitable means.

In this manner, the analyte of interest is brought into contact with a suitable enzyme and a coenzyme, where the enzyme is present in catalytic amounts. The coenzyme is physicochemically altered (i.e., oxidized or reduced) by the enzymatic reaction, and the reaction can be electrochemically or photometrically detected. Calibration provides a direct link between the measurement value and the analyte concentration.

Diagnostic test elements are known and can be distinguished by a temporally limited storage life and by specific requirements on the environment, such as cooling or dry storage, so as to achieve this storage life. Therefore, in tests carried out by an end user, such as in blood glucose self-monitoring, unnoticed incorrect storage of the measurement system could lead to incorrect results, which cannot really be recognized by the user and may potentially lead to incorrect treatment of the relevant illness.

The incorrect results are primarily due to substances used in diagnostic test elements of this type, in particular enzymes, coenzymes and/or mediators, which generally react sensitively to humidity, heat and/or light and become deactivated over time. Among other things, this has the result that service lives, determined during manufacturing of a testing chemistry prepared in aqueous solution must not be exceeded, and the testing chemistry applied to a suitable carrier cannot be kept for too long before the next manufacturing step.

To take into account these limitations imposed by the testing chemistry, the size of the batches of a testing chemistry used for making diagnostic test elements is usually limited. In this way, it can be ensured that an individual batch of the testing chemistry is substantially homogeneous, and further that the substances contained in the testing chemistry still have sufficiently high activity for subsequently detecting an analyte of interest, even after the processing thereof to form a diagnostic test element.

Meanwhile, since each batch of a testing chemistry used to produce diagnostic test elements usually receives its own batch coding for reasons of quality, this usually leads to the problem that a single batch coding merely covers a small number of diagnostic test elements. To code a large number of diagnostic test elements, a large number of different batch codings have to be created, which involves considerable additional temporal, technological and financial outlay.

For the foregoing reasons, there is a need for improved methods of making test elements in which one can prepare large, homogeneous batches of a testing chemistry to be used, to thereby provide a high homogeneity of batches produced in succession of diagnostic test elements, to make it possible to use a single batch coding for a large number of diagnostic test elements, and further to provide the option of coding individual diagnostic test elements.

### BRIEF SUMMARY

An inventive concept described herein includes simplifying the producing, checking, packaging and handling of diagnostic test elements by using an analytical chemical reagent having a high stability in relation to humidity, heat and light. This inventive concept can be incorporated into exemplary analytical chemical reagents that include a combination of a coenzyme-dependent enzyme and an artificial coenzyme. Further, the use of an analytical chemical reagent of this type results in temporal and financial advantages, which are of major importance especially in the industrial-scale production of diagnostic test elements. This inventive concept can be incorporated into exemplary methods and test elements as described herein and in more detail below.

For example, methods are provided that include a step of (a) providing a batch of an analytical chemical reagent including a coenzyme-dependent enzyme and an artificial coenzyme; a step of (b) coating a first carrier with a first part of the batch of the analytical chemical reagent; a step of (c) splitting up the coated first carrier into a plurality of first diagnostic test elements; a step of (d) creating a batch coding for the batch of analytical chemical reagent using at least one of the first diagnostic test elements; a step of (e) coating a second carrier with a second part of the batch of the analytical chemical reagent; and a step of (f) splitting up the coated second carrier into a plurality of second diagnostic test elements.

The methods optionally can include a step of (g) checking and packaging the second diagnostic test elements, where the second diagnostic test elements are provided with the batch coding created in step (d) before the coated second carrier is split up.

In the methods, the coenzyme-dependent enzyme can be a flavin-, nicotinamide- or pyrroloquinoline-quinone-dependent oxidoreductase. Alternatively, the coenzyme-dependent enzyme can be a flavin-, nicotinamide- or pyrroloquinoline-quinone-dependent dehydrogenase, especially a NAD(P)/NAD(P)H-dependent dehydrogenase.

The artificial coenzyme can be an artificial NAD(P)/NAD(P)H compound such as carbaNAD, or a compound of formula (I):

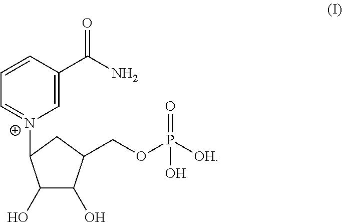 [SMILES: NC(=O)c1ccc[n+](C2CC(COP(=O)(O)O)C(O)C2O)c1]

In some instances, there can be a period of at least about 36 hours to at least about 48 hours between steps (a) and (e) and/or a period of at least about 20 days to at least about 30 days between steps (e) and (f).

Also provided are methods of increasing batch size and/or batch homogeneity of diagnostic test elements. The methods can include a step of using an analytical chemical reagent as described herein when producing a batch of diagnostic test elements, where the analytical chemical reagent increases the batch size and/or batch homogeneity, and where individual diagnostic test elements of the batch are substantially identical in each case.

In some instances, the batch size is increased by a factor of 2, by a factor of 3, or even by a factor of 5 when compared to an analytical chemical reagent that includes a coenzyme-dependent enzyme and a native coenzyme.

In view of the foregoing, the diagnostic products are provided that include (a) at least one diagnostic test element, where the diagnostic test element includes (i) a carrier, (ii) an analytical chemical reagent as described herein which is applied to the carrier in the form of a layer, and (iii) a batch coding; and (b) optionally at least one needle element for scratching the skin.

These and other advantages, effects, features and objects of the inventive concept will become better understood from the description that follows. In the description, reference is made to the accompanying drawings, which form a part hereof and in which there is shown by way of illustration, not limitation, embodiments of the inventive concept.

### BRIEF DESCRIPTION OF THE DRAWINGS

The advantages, effects, features and objects other than those set forth above will become more readily apparent when consideration is given to the detailed description below. Such detailed description makes reference to the following drawings, wherein:

FIG. 1 shows activity of glucose dehydrogenase double mutant GlucDH_E96G_E170K when the enzyme is stored in the presence of carbaNAD for a period of 52 weeks at a temperature of 5° C. or 35° C. and a relative air humidity of 0% (desiccant), 75% or 85%.

FIG. 2 shows carbaNAD (cNAD) content when the coenzyme is stored in the presence of glucose dehydrogenase double mutant GlucDH_E96G_E170K for a period of 52 weeks at a temperature of 5° C. or 35° C. and a relative air humidity of 0% (desiccant), 75% or 85%.

FIG. 3 shows amino acid sequences of the glucose dehydrogenase double mutants GlucDH_E96G_E170K (GlucDH-Mut1; SEQ ID NO:1) and Gluc DH_E170K_K252L (GlucDH-Mut2; SEQ ID NO:2), which were obtained by mutating wild type glucose dehydrogenase from Bacillus subtilis.

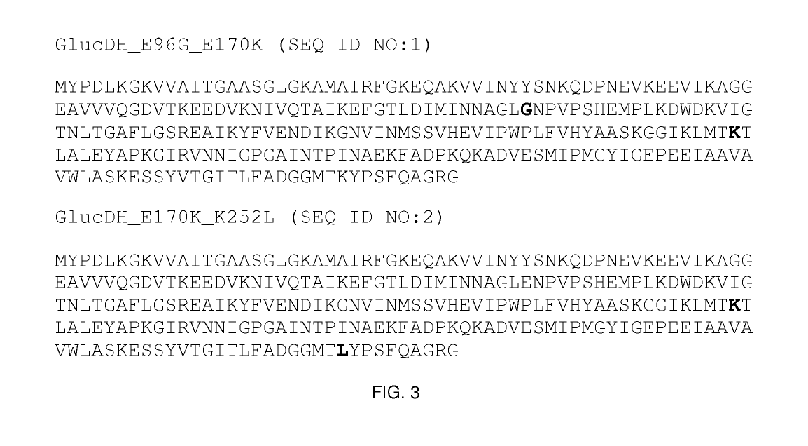

FIG. 4 shows stability of lactate dehydrogenase (LDH) in 2.5% NaCl-containing K/NaP₂O₇ at pH 8.0 and a temperature of 40° C. or 50° C. The stability is shown in the presence or absence of co-factors NAD and carbaNAD (cNAD).

FIG. 5 shows stability of glutamate dehydrogenase (GIDH) in 2.5% NaCl-containing K/NaP₂O₇ at pH 8.0 and a temperature of 40° C. or 50° C. The stability is shown in the presence or absence of co-factors NAD and carbaNAD (cNAD).

Corresponding reference characters indicate corresponding parts throughout the several views of the drawings.

While the inventive concept is susceptible to various modifications and alternative forms, exemplary embodiments thereof are shown by way of example in the drawings and are herein described in detail. It should be understood, however, that the description of exemplary embodiments that follows is not intended to limit the inventive concept to the particular forms disclosed, but on the contrary, the intention is to cover all advantages, effects, features and objects falling within the spirit and scope thereof as defined by the embodiments described herein and the claims below. Reference should therefore be made to the embodiments described herein and claims below for interpreting the scope of the inventive concept. As such, it should be noted that the embodiments described herein may have advantages, effects, features and objects useful in solving other problems.

### DESCRIPTION OF EXEMPLARY EMBODIMENTS

The methods and test elements now will be described more fully hereinafter with reference to the accompanying drawings, in which some, but not all embodiments of the inventive concept are shown. Indeed, the methods and test elements may be embodied in many different forms and should not be construed as limited to the embodiments set forth herein; rather, these embodiments are provided so that this disclosure will satisfy applicable legal requirements.

Likewise, many modifications and other embodiments of the methods and test elements described herein will come to mind to one of skill in the art to which the disclosure pertains having the benefit of the teachings presented in the foregoing descriptions and the associated drawings. Therefore, it is to be understood that the methods and test elements are not to be limited to the specific embodiments disclosed and that modifications and other embodiments are intended to be included within the scope of the appended claims. Although specific terms are employed herein, they are used in a generic and descriptive sense only and not for purposes of limitation.

Unless defined otherwise, all technical and scientific terms used herein have the same meaning as commonly understood by one of skill in the art to which the disclosure pertains. Although any methods and materials similar to or equivalent to those described herein can be used in the practice or testing of the methods and test elements, the preferred methods and materials are described herein.

Moreover, reference to an element by the indefinite article “a” or “an” does not exclude the possibility that more than one element is present, unless the context clearly requires that there be one and only one element. The indefinite article “a” or “an” thus usually means “at least one.” Likewise, the terms “have,” “comprise” or “include” or any arbitrary grammatical variations thereof are used in a non-exclusive way. Thus, these terms may both refer to a situation in which, besides the feature introduced by these terms, no further features are present in the entity described in this context and to a situation in which one or more further features are present. For example, the expressions “A has B,” “A comprises B” and “A includes B” may refer both to a situation in which, besides B, no other element is present in A (i.e., a situation in which A solely and exclusively consists of B) or to a situation in which, besides B, one or more further elements are present in A, such as element C, elements C and D, or even further elements.

Methods

Methods incorporating the inventive concept can include method of producing diagnostic test elements. The methods can include the steps described herein, and these steps may be, but not necessarily, carried out in the sequence as described. Other sequences, however, also are conceivable. Furthermore, individual or multiple steps may be carried out either in parallel and/or overlapping in time and/or individually or in multiply repeated steps. Moreover, the methods may include additional, unspecified steps.

The methods generally begin by a step of (a) providing a batch of an analytical chemical reagent that includes a coenzyme-dependent enzyme and an artificial coenzyme.

With respect to the coenzyme-dependent enzyme, it generally is a dehydrogenase. Examples of dehydrogenases that can be used herein include, but are not limited to, alcohol dehydrogenase (E.C. 1.1.1.1; E.C. 1.1.1.2), L-amino acid dehydrogenase (E.C. 1.4.1.5), glucose dehydrogenase (E.C. 1.1.1.47), glucose-6-phosphate dehydrogenase (E.C. 1.1.1.49), glycerine dehydrogenase (E.C. 1.1.1.6), 3-hydroxybutyrate dehydrogenase (E.C. 1.1.1.30), lactate dehydrogenase (E.C. 1.1.1.27; E.C. 1.1.1.28), malate dehydrogenase (E.C. 1.1.1.37), glutamate dehydrogenase (E.C. 1.4.1.2; E.C. 1.4.1.3; E.C. 1.4.1.4) and sorbitol dehydrogenase. In some instances, the dehydrogenase is glucose dehydrogenase (E.C. 1.1.1.47), glucose-6-phosphate dehydrogenase (E.C. 1.1.1.49), lactate dehydrogenase (E.C. 1.1.1.27; E.C. 1.1.1.28), or glutamate dehydrogenase (E.C. 1.4.1.2; E.C. 1.4.1.3; E.C. 1.4.1.4).

Although native coenzyme-dependent enzymes can be used, mutant coenzyme-dependent enzymes also can be used. As used herein, “mutated” or “mutant” coenzyme-dependent enzyme means a genetically altered variant of a native coenzyme-dependent enzyme (e.g., wild-type enzyme), the variant having the same number of amino acids as the wild-type enzyme but a different amino acid sequence that thus differs from the wild type enzyme in at least one amino acid. Generally, the mutant coenzyme-dependent enzyme has an increased thermal and/or hydrolytic stability when compared to the wild-type enzyme.

Mutant coenzyme-dependent enzymes can be obtained by mutating a native coenzyme-dependent enzyme originating from any biological source. As used herein, “biological source” means both prokaryotes and eukaryotes. The introduction of the mutation(s) may be localized or non-localized; however, in some instances the localized mutations result from recombinant methods known in the field, where at least one amino acid exchange is introduced within the amino acid sequence of the native enzyme.

In some instances, the mutant coenzyme-dependent enzyme is a mutated glucose dehydrogenase (E.C. 1.1.1.47) or a mutated glucose-6-phosphate dehydrogenase (E.C. 1.1.1.49). Examples of specific mutated glucose dehydrogenases can be found in, for example, Int'l Patent Application Publication Nos. WO 2005/045016 and WO 2011/020856; Baik et al. (2005) Appl. Environ. Microbiol. 71:3285; and Vasquez-Figueroa et al. (2007) Chem Bio Chem 8:2295. In other instances, the coenzyme-dependent enzyme is a mutated glucose dehydrogenase having an amino acid sequence as shown in SEQ ID NO:1 (GlucDH_E96G_E170K) or as shown in SEQ ID NO:2 (GlucDH_E170K_K252L).

Aside from the coenzyme-dependent enzyme, the analytical chemical reagent also includes at least one artificial coenzyme. As used herein, “artificial coenzyme” means a coenzyme that is chemically altered with respect to a native coenzyme and that at atmospheric pressure has a higher stability than the native coenzyme against humidity, temperatures in a region of about 0° C. to about 50° C., acids and bases in a range of pH 4 to pH 10, and/or nucleophiles such as alcohols or amines, and can thus produce its effect for a longer time when compared to the native coenzyme under identical ambient conditions.

As used herein, “about” means within a statistically meaningful range of a value or values such as, for example, a stated concentration, length, width, height, angle, weight, molecular weight, pH, sequence identity, time frame, temperature or volume. Such a value or range can be within an order of magnitude, typically within 20%, more typically within 10%, and even more typically within 5% of a given value or range. The allowable variation encompassed by “about” will depend upon the particular system under study, and can be readily appreciated by one of skill in the art.

In some instances, the artificial coenzyme has a higher hydrolytic stability than the native coenzyme, complete hydrolytic stability under test conditions being particularly advantageous. The artificial coenzyme may have a lower binding constant than the native coenzyme for the coenzyme-dependent enzyme such as, for example, a binding constant reduced by a factor of 2 or more.

Examples of artificial coenzymes include, but are not limited to, artificial NAD(P)/NAD(P)H compounds, which are chemical derivatives of native nicotinamide adenine dinucleotide (NAD/NADH) or native nicotinamide adenine dinucleotide phosphate (NADP/NADPH), or the compound of formula (I):

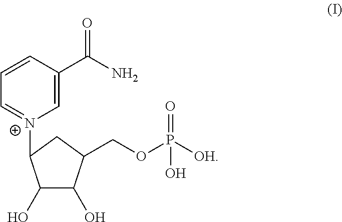 [SMILES: NC(=O)c1ccc[n+](C2CC(COP(=O)(O)O)C(O)C2O)c1]

If the artificial coenzyme is an artificial NAD(P)/NAD(P)H compound, the artificial NAD(P)/NAD(P)H compound can include a 3-pyridine carbonyl or 3-pyridine thiocarbonyl radical, which is linked via a linear or cyclic organic radical (e.g., via a cyclic organic radical having a phosphorous-containing radical such as phosphate radical) without glycosidic bonding.

Alternatively, the artificial coenzyme can be a compound of general formula (II):

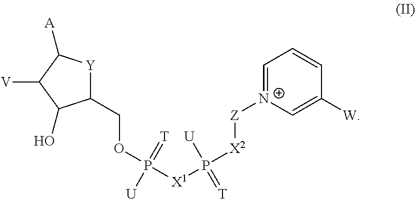

in which A=adenine or an analogue thereof,
T=O, S independently in each case,
U=OH, SH, BH₃⁻, BCNH₂⁻ independently in each case,
V=OH or a phosphate group or two groups which form a cyclic phosphate group independently in each case,
W=COOR, CON(R)₂, COR, CSN(R)₂, having R=H or C₁-C₂ alkyl independently in each case,
X¹, X²=O, CH₂, CHCH₃, C(CH₃)₂, NH, NCH₃, independently in each case,

### Y=NH, S, O, CH₂, and

Z=a linear or cyclic organic radical, with the proviso that Z and the pyridine radical are not linked by a glycosidic bond, or a salt or reduced form thereof.

In the compounds of formula (II), Z can be a linear radical having 4-6 C atoms, especially having 4 C atoms, in which 1 or 2 C atoms are optionally substituted with one or more heteroatoms selected from O, S and N, or a radical including a cyclic group having 5 or 6 C atoms, which optionally contains a heteroatom selected from O, S and N and optionally one or more substituents, and a radical CR⁴₂, in which C⁴₂ is bonded to the cyclic group and to X², having R⁴=H, F, Cl, CH₃ independently in each case.

In certain instances, Z is a saturated or unsaturated carbocyclic or heterocyclic five-membered ring, such as a radical of general formula (III),

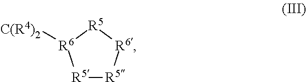

it being possible for there to be a single or double bond between R^{5′} and R^{5″}, in which
R⁴=H, F, Cl, CH₃ independently in each case,

### R⁵=CR⁴₂,

R^{5′}=O, S, NH, NC₁-C₂ alkyl, CR⁴₂, CHOH, CHOCH₃, and R^{5″}=CR⁴₂, CHOH, CHOCH₃ if there is a single bond between R^{5′} and R^{5″},
R^{5′}=R^{5″}=CR⁴ if there is a double bond between R^{5′} and R^{5″}, and
R⁶, R^{6′}=CH or CCH₃ independently in each case.

The compounds of general formula (II) can include an adenine analogue. As used herein, “adenine analogue” means a chemical derivative of native adenine which produces the same pharmacological effect as adenine in the human body. Specific examples of adenine analogues include, but are not limited to, C₈ and N₆-substituted adenine, 7-deazaadenine, 8-azaadenine, 7-deaza-8-azaadenine and formycin, it being possible for the 7-deaza variants to be substituted with halogen, C₁₋₆ alkynyl, C₁₋₆ alkenyl or C₁₋₆ alkyl in the 7 position.

Alternatively, the compounds can contain adenosine analogues that, instead of ribose, include 2-methoxydeoxyribose, 2′-fluorodeoxyribose, hexitol, altritol or polycyclic analogues, such as bicyclo-, LNA- and tricyclo-sugars. Additionally, in the compounds of formula (II), (di-)phosphate oxygens also may be isotronically substituted (e.g., O⁻ with S⁻ or BH₃, O with NH, NCH₃ or CH₂, and ═O with ═S). In compounds of formula (II), W can be CONH₂ or COCH₃.

In compounds of formula (III), R⁵ can be CH₂. It is further contemplated that R^{5′} can be selected from CH₂, CHOH and NH. In certain instances, R^{5′} and R^{5″} are each CHOH. Alternatively, R^{5′} can be NH and R^{5″} can be CH₂. In specific instances, the compound can be of formula (III) in which R⁴=H, R⁵=CH₂, R^{5′}=R^{5″}=CHOH and R⁶=R^{6′}=CH.

In some instances, the artificial coenzyme is carbaNAD. See, Slama & Simmons (1988) Biochem. 27:183-193; and Slama & Simmons (1989) Biochem. 28:7688-7694. Other stable coenzymes that may be used in the methods are disclosed in Int'l Patent Application Publication Nos. WO 98/33936, WO 01/49247 and WO 2007/012494 U.S. Pat. No. 5,801,006; U.S. patent application Ser. No. 11/460,366; and Blackburn et al. (1996) Chem. Comm. 2765-2766.

Aside from the coenzyme-dependent enzyme and the artificial coenzyme, the analytical chemical reagent also can include other substances used for qualitative analysis and/or quantitative determination of analytes, such as a mediator and/or an optical indicator. As used herein, “mediator” means a chemical compound that increases reactivity of a reduced coenzyme obtained by reaction with the analyte and that makes transfer of electrons to a suitable optical indicator or to an optical indicator system. Examples of mediators include, but are not limited to, azo compounds, nitrosoanilines, quinones and phenazines.

As used herein, “optical indicator” means any desired substance that may be used, that is reducible, and that upon reduction undergoes a visually detectable and/or machine-detectable change in the optical properties thereof, such as color, fluorescence, remission, transmission, polarisation and/or refractive index. Examples of optical indicators include, but are not limited to, reducible heteropolyacids such as 2,18-phosphomolybdic acid. Alternatively, quinones such as resazurin, dichlorophenolindophenol and/or tetrazolium salts may be used as optical indicators.

The methods also can include a step of (b) coating a first carrier with a first part of the batch of the analytical chemical reagent. Once a batch of the analytical chemical reagent has been provided, which can be in liquid form such as in the form of a suspension, a first part of this batch is applied to a first carrier such as a film or an injection-moulded part and subsequently dried, causing the first carrier to be coated with the first batch of the analytical chemical reagent. While both this step and the following processing steps may only take a short time when a conventional testing chemistry is used, which is sensitive to humidity, heat and/or light, the use of a combination of coenzyme-dependent enzyme and artificial coenzyme and the long-term stability of the testing chemistry that is achieved as a result mean that it is possible to refrain from immediate further processing of the entire batch of the analytical chemical reagent, and this leads to considerable production-related advantages.

Because of the insensitivity of the analytical chemical reagent used herein towards humidity, heat and light, much larger batches of the analytical chemical reagent can be produced without the testing chemistry being impaired by the environment during the production or processing process. Accordingly, a larger number of carriers also can be coated with a single batch of the analytical chemical reagent, ultimately making it possible to produce a larger batch of diagnostic test elements, which in addition is more homogeneous than a single batch of diagnostic test elements which includes conventional testing chemistry.

Further, the analytical chemical reagent disclosed herein has the advantage that a plurality of batches of diagnostic test elements can be produced using a single batch thereof, and are thus homogeneous with one another. This ensures that even for a large batch of the analytical chemical reagent, the first diagnostic test elements produced therefrom and the last diagnostic test elements produced therefrom are identical or merely vary within narrow boundaries. By contrast, if conventional testing chemistry is used, small batches of diagnostic test elements have to be produced, in such a way that the distribution of the reactivity of the analytical chemical reagent, which is impaired by environmental influences, still varies within the acceptable range.

The methods also can include a step of (c) splitting up the coated first carrier into a plurality of first diagnostic test elements, and a step of (d) creating a batch coding for the batch of analytical chemical reagent using at least one of the first diagnostic test elements. After the coating, the coated first carrier is split up into a plurality of first diagnostic test elements by suitable techniques (preliminary batch), and a batch coding for the entire batch of the analytical chemical reagent is created using at least one of the first diagnostic test elements. The batch coding created in this context preferably contains a mathematical equation which specifies the potentially temperature-dependent relationship between the respective amount of an analyte to be determined and the resulting signal, which can be measured for example optically or electrochemically.

In principle, any code that appears appropriate to one of skill in the art for coding diagnostic test elements may be used as the batch coding, and reliable tracking of the diagnostic test elements provided with the code is made possible using suitable means. In some instances, an optically and/or electronically readable code, such as a barcode or an RFID transponder, is used as the batch coding. In certain instances, a barcode can be used, which may be made one-dimensional or two-dimensional, may be configured in black and white, greyscale or color, and may include a hologram if desired. Alternatively, especially for producing test elements for electrochemical detection, there is the option of implementing a coding by electrically contacting the individual diagnostic test elements.

The methods also can include a step of (e) coating a second carrier with a second part of the batch of the analytical chemical reagent, and then a step of (f) splitting up the coated second carrier into a plurality of second diagnostic test elements. After the batch coding is created, a second carrier can be coated, in particular a film or an injection-moulded part, with a second part of the batch of the analytical chemical reagent. After applying the analytical chemical reagent to the second carrier and subsequent drying, a coated second carrier is obtained, from which a plurality of second diagnostic test elements are obtained by splitting it up, which may undergo checking if required and be suitably packaged after passing the quality control.

In some instances, the second diagnostic test elements are coded before the coated second carrier is split up. In this context, the second diagnostic test elements are each individually provided with the batch coding created using the preliminary batch (in other words using the first diagnostic test elements), and this can be done by applying the batch coding to the second carrier before the second carrier is coated with the second part of the batch of the analytical chemical reagent. This has major financial and production-related advantages, since a single batch coding is sufficient to code a greater number of diagnostic test elements, by a factor of 2, by a factor of 3, or even by a factor of 5, than when conventional testing chemistry is used.

At the same time, by selective application of the batch coding to the second carrier, it is possible to code each individual diagnostic element, and this is generally not possible when conventional testing chemistry is used. As stated above, creating a batch coding usually requires the production of a preliminary batch. However, the subsequent coding of diagnostic test elements using the batch coding thus obtained has to take place in an earlier step, and in any case before the respective carrier is split up into the diagnostic test elements, so as to be economically viable and technically feasible.

The methods optionally can include a step of (g) checking and packaging the second diagnostic test elements, where the second diagnostic test elements are provided with the batch coding created in step (d) before the coated second carrier is split up.

In the methods, creating a batch coding using a preliminary batch still requires a break in production. Meanwhile, for conventional testing chemistry, which has a low stability towards humidity, heat and/or light, a break in production of this type to create a batch coding is not possible, since the testing chemistry is impaired in the event of a longer storage time or a processing process which runs for longer. The batch coding of a preliminary batch of this type would therefore differ greatly and in an irreproducible manner from the batch coding of a subsequently produced batch of diagnostic test elements.

As a result of coding of individual diagnostic test elements, it is not necessary to use ROM keys or for the user to input code numbers. The advantage of coding individual diagnostic test elements over coding a whole magazine (e.g., a box containing test strips) can be seen in elements where the user is supposed to carry out the coding himself, but neglects to do so for reasons of convenience, forgetfulness or unawareness. Coding individual diagnostic test elements thus prevents the risk of the user carrying out a measurement using a non-matching code (e.g., using an earlier batch of the diagnostic element).

Because of the insensitivity of the analytical chemical reagent towards humidity, heat and light, a batch of the analytical chemical reagent does not have to be processed further immediately after the production thereof so as to prevent a change in the chemical and/or physical properties thereof. Instead, the methods described herein make it possible for there to be a period of several hours to several weeks between the initial preparation of a batch of the analytical chemical reagent and the completed processing thereof to form diagnostic test elements, and this has temporal and production-related advantages.

In some instances in the methods herein, there is a period of at least about 36 hours to at least about 48 hours between preparing the batch of the analytical chemical reagent and coating the second carrier with a second part of the batch of the analytical chemical reagent after the batch coding is created. Further, for reasons relating to production, there can be a period of at least about 20 days to at least about 30 days between coating the second carrier with the second part of the batch of the analytical chemical reagent and splitting up the coated second carrier into a plurality of second diagnostic test elements.

Specifically, the period between preparing the batch of the analytical chemical reagent and coating the second carrier and/or between coating the second carrier and splitting it up into a plurality of second diagnostic test elements is preferably selected in such a way that the batch of the analytical chemical reagent undergoes substantially no change in the chemical and/or physical properties thereof, and accordingly makes it possible to produce diagnostic test elements which ensure determination of analytes within the legally permissible range.

As used herein, “substantially no change in the chemical and/or physical properties” means a decrease in the activity of the coenzyme-dependent enzyme or a decrease in the content of artificial coenzyme in the analytical chemical reagent of less than about 40%, of less than about 30% or even of less than about 20% based on the activity of the coenzyme-dependent enzyme or based on the content of artificial coenzyme in the analytical chemical reagent immediately after the production thereof.

The first diagnostic test elements and second diagnostic test elements produced by the methods disclosed herein may be formed identically or differently, but are preferably formed identically. The diagnostic test elements may in principle be of any physical shape familiar to one of skill in the art, which is suitable for determining the presence and/or the amount of an analyte in a sample, and they each include at least one test field that can be brought into contact with a sample containing the analyte and that makes qualitative and/or quantitative determination of the analyte possible using suitable means.

Examples of first diagnostic test elements and/or second diagnostic test elements include in particular test elements such as test strips, test bands and test discs, from which diagnostic test elements based thereon, such as test strip magazines and band magazines, can be produced if required. Suitable test strip magazines include blister magazines, leporello magazines, disc magazines, stack magazines, drum magazines and rotating magazines, which are disclosed in, for example, EP Patent Nos. 0 951 939, 1 022 565 and 1 736 772, as well as Int'l Patent Application Publication Nos. WO 2005/104948 and WO 2010/094427. Band magazines are disclosed in DE 10 2005 013 685, EP Patent No. 1 739 432 and Int'l Patent Application No. WO 2004/047642.

In some instances, the first diagnostic test elements and/or the second diagnostic test elements each include a plurality of test fields such as, for example, at least about 10 individual test fields, at least about 25 individual test fields or even at least about 50 individual test fields. In certain instances, the individual test fields are each arranged at a distance of a few millimeters to a few centimeters from one another, for example at a distance of <2.5 cm.

Moreover, and in some instances, each of the test fields of the first test diagnostic test elements and/or the second diagnostic test elements is enclosed at least in part by a hydrophobic edge and/or stored in its own substantially closed chamber. The use of a hydrophobic edge is advantageous in cases where a user has to apply a sample of the analyte to be determined to a test field of the respective diagnostic element manually, and the sample is to be prevented from overflowing to adjacent test fields or entering a device needed for measuring the diagnostic test element.

Diagnostic test elements in which each test field is mounted in its own substantially closed chamber are used, for example, in fully automatic measurement systems. Measurement systems of this type include a plurality of test fields, which may be arranged for example annularly side by side on a rotatable plate, as well as a large number of needle elements, formed for scratching the skin, in a magazined form. Although manual application of the sample to a test field by the user is not necessary in these cases (the system does this automatically), the individual test fields have to be located in mutually separated chambers for reasons of hygiene. As used herein, “substantially closed chamber” means that the chamber walls may be permeable to air and/or water, but do not allow dust particles to enter the chamber and thus make dust-free storage of the test fields possible.

If the test fields of a diagnostic test element contain conventional testing chemistry, which has a low stability towards humidity, heat and/or light, the hydrophobic edge that encloses the test fields at least in part or the chambers that surround the test fields have to be produced by a complicated and technically demanding method. Meanwhile, because the analytical chemical reagent described herein is stable among other things towards light, and in particular towards UV light, the diagnostic test elements disclosed herein also may include light-curable or UV-curable materials.

Therefore, in some instances, the methods described herein provide that the hydrophobic edge that encloses the test fields of a diagnostic test element and/or the chamber walls of the chambers surrounding the individual test fields are formed of a UV-curable material. Specific examples of UV-curable materials that may be made use of in this context are known to one of skill in the art and include among other things epoxide and acrylate adhesives, but are not limited thereto. Further, it is possible to use light selectively to generate a layer of the analytical chemical reagent on the diagnostic test elements.

The stability of the analytical chemical reagents described herein towards light further results in significant advantages in relation to the production and any quality control of diagnostic test elements produced therefrom. Thus, on the one hand no measures that are demanding in terms of production, for protecting the diagnostic test elements from light, need to be taken. In this way, it is possible to simplify the production considerably and simultaneously to reduce the production costs.

On the other hand, diagnostic test elements that are coated with the analytical chemical reagents described herein may be analyzed for possible defects or for an uneven distribution of the coating by optical methods. Accordingly, in some instances, the methods described herein additionally include checking the thickness and/or homogeneity of the layer of the analytical chemical reagent on the second diagnostic test elements.

At the same time, diagnostic test elements that are coated with an analytical chemical reagent as described herein also have a high stability towards humidity. Consequently, diagnostic test elements produced by the methods described herein and subsequently packaged are not subjected to water vapor tightness checking, making it possible to speed up the manufacturing process as a whole and to reduce the production costs.

The diagnostic test elements produced by the methods described herein can be used for the qualitative and/or quantitative determination of any biological or chemical substance that is optically or electrochemically traceable. In some instances, the analyte is selected from malic acid, alcohol, ascorbic acid, cholesterol, glucose, glycerol, urea, 3-hydroxybutyrate, lactic acid, pyruvate and triglycerides, especially glucose.

The analyte to be determined may be from any source, but typically can be contained in a bodily fluid, including, but not limited to, whole blood, plasma, serum, lymph fluid, bile fluid, cerebrospinal fluid, extracellular tissue fluid, urine and glandular secretions such as saliva or sweat. Meanwhile, the presence and/or amount of an analyte in a sample can be determined by means of the analytical reagents described herein.

Methods incorporating the inventive concept also can include using an analytical chemical reagent as described herein to increasing batch size and/or batch homogeneity when producing diagnostic test elements, where the reagent includes a coenzyme-dependent enzyme and an artificial coenzyme to increase the batch size and/or batch homogeneity, and where the individual diagnostic elements of a batch being substantially identical in each case.

In some instances, by using at least one analytical chemical reagent as described herein, the batch size can be increased by a factor of about 2, by a factor of about 3, or even by a factor of about 5 when compared to an analytical chemical reagent that includes a coenzyme-dependent enzyme and a native coenzyme.

With respect to the analytical chemical reagent and the diagnostic elements, reference is made to the statements above and below, respectively.

Diagnostic Devices

Devices incorporating the inventive concept can include diagnostic devices. The devices can include (a) at least one diagnostic test element, and (b) optionally at least one needle element for scratching the skin.

Briefly, the diagnostic test element can include (i) a carrier; (ii) an analytical chemical reagent as described herein, which includes a coenzyme-dependent enzyme and an artificial coenzyme and which is applied to the carrier in the form of a layer; and (iii) a batch coding.

In some instances, aside from the analytical chemical reagent, the diagnostic device also can include a needle element for scratching the skin, which can include a sterilizable material, such as metal or plastics material. So as to make it possible to transfer the bodily fluid to be analyzed, such as blood, to the diagnostic test element, the needle element can include a capillary duct, by means of which a sufficient amount of the sample can be taken and be applied to a test field of the diagnostic test element using capillary forces.

In some instances, the diagnostic test element (including the carrier, analytical chemical reagent and batch coding), reference is made to the statements made in connection with the description of the methods above.

### EXAMPLES

The inventive concept will be more fully understood upon consideration of the following non-limiting examples, which are offered for purposes of illustration, not limitation.

### Example 1

A mixture of glucose dehydrogenase double mutant GlucDH_E170K_K252L and carbaNAD was placed in storage (a) at a temperature of 5° C. and a relative air humidity of 0% (in other words in the presence of a desiccant), (b) at a temperature of 5° C. and a relative air humidity of 75%, (c) at a temperature of 35° C. and a relative air humidity of 0%, and (d) at a temperature of 35° C. and a relative air humidity of 85%, in each case for a period of 52 weeks.

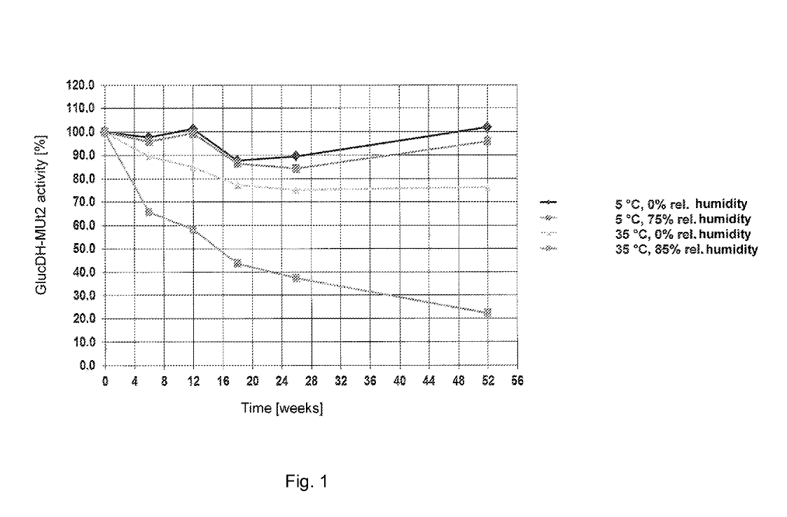

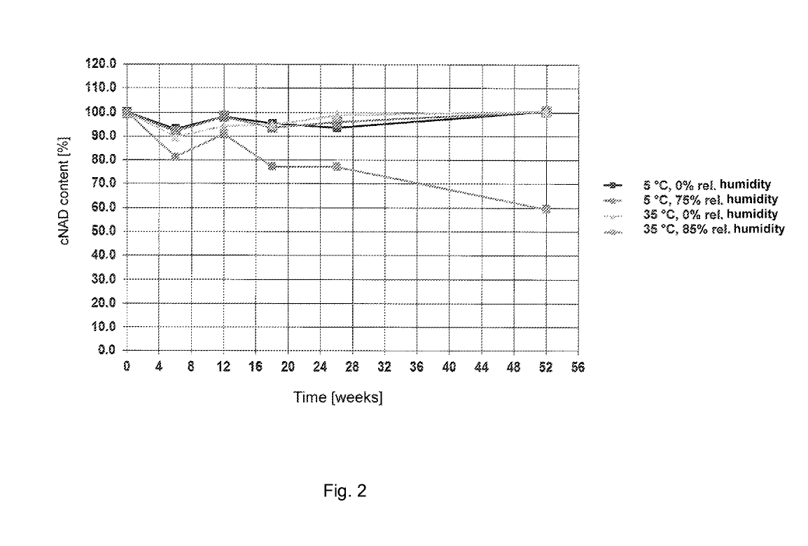

Subsequently, the activity of the glucose dehydrogenase double mutant GlucDH_E170K_K252L and the content of carbaNAD in the mixture of enzyme and artificial coenzyme were determined at regular intervals. A graphical representation of the results of these determinations is shown in FIGS. 1-2.

In the context of these measurements, it was found that the activity of the enzyme after storing for 52 weeks at a temperature of 5° C. and a relative air humidity of 0% is almost 100%. After the mixture is placed in storage at a temperature of 5° C. and a relative air humidity of 75%, an enzyme activity of approximately 95%, based on the initial value, is still measured (see, FIG. 1).

If the enzyme was placed in storage for 52 weeks at a temperature of 35° C. and a relative air humidity of 0%, the residual activity was approximately 75%. After the mixture of enzyme and artificial coenzyme was placed in storage at a temperature of 35° C. and a relative air humidity of 85%, an enzyme activity of approximately 20%, based on the initial value, was observed after 52 weeks.

As regards the stability of carbaNAD in the mixture of enzyme and artificial coenzyme, FIG. 2 shows that the content of carbaNAD after storage for 52 weeks at a temperature of 5° C., both at a relative air humidity of 0% and at a relative air humidity of 75%, is almost 100%, based on the initial value.

Even if the mixture of enzyme and artificial coenzyme was placed in storage for 52 weeks at a temperature of 35° C. and a relative air humidity of 0%, the content of carbaNAD was approximately 100% after this time. If the mixture was placed in storage at a temperature of 35° C. and a relative air humidity of 85%, this still resulted in a coenzyme content of approximately 60%, based on the initial value, after 52 weeks.

These data show that carbaNAD is stable for a long period at elevated temperatures and/or at elevated relative air humidity. At the same time, it is clear that the glucose dehydrogenase double mutant GlucDH_E170K_K252L has a stability in the presence of carbaNAD, and this leads in particular to advantages in the production and processing of diagnostic elements for glucose determination.

### Example 2

Lactate dehydrogenase (LDH) was exposed to temperatures of 40° C. and 50° C. in 2.5% NaCl-containing K/NaP₂O₇ solution. Subsequently, the activity of the LDH was analysed at the start and after 3, 21 and 45 hours. The measurement was taken in the presence and absence of the cofactors NAD and carbaNAD (cNAD).

These measurements showed that the activity of the enzyme greatly decreases at an elevated temperature of 50° C. in the absence of a cofactor and in the presence of the cofactor NAD, whilst the stability of LDH in the presence of the cofactor carbaNAD was still in the region of the initial value even after several days. The results are summarized in Table 1 below:

TABLE 1

| Sample | 0 h | 3 h, 40° C. | 21 h, 40° C. | 45 h, 40° C. |
|---|---|---|---|---|
| Without | 100% | 115% | 107% | 119% |
| cofactor | | | | |
| With NAD | 100% | 113% | 103% | 112% |
| With cNAD | 100% | 106% | 110% | 117% |
| Sample | 0 h | 3 h, 50° C. | 21 h, 50° C. | 45 h, 50° C. |
| Without | 100% | 94% | 64% | 37% |
| cofactor | | | | |
| With NAD | 100% | 104% | 74% | 47% |
| With cNAD | 100% | 101% | 101% | 101% |

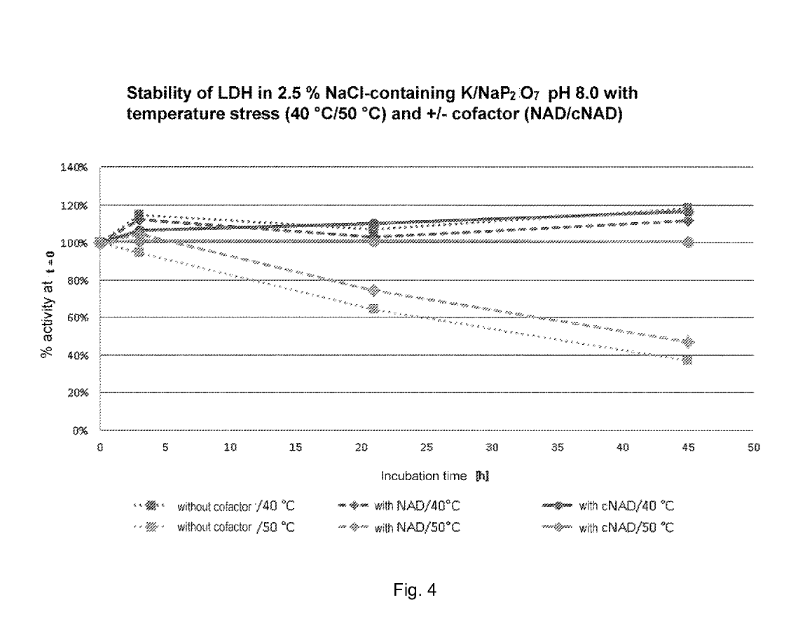

A graphical representation of the results of these determinations is shown in FIG. 4.

### Example 3

Glutamate dehydrogenase (GIDH) was exposed to temperatures of 40° C. and 50° C. in 2.5% NaCl-containing K/NaP₂O₇ solution Subsequently, the activity of the GIDH was analysed at the start and after 3, 24 and 45 hours. The measurement was taken in the presence and absence of the cofactors NAD and carbaNAD (cNAD).

These measurements showed that the activity of the enzyme greatly decreases at an elevated temperature of 50° C. in the absence of a cofactor and in the presence of the cofactor NAD, whilst the stability of the glutamate dehydrogenase in the presence of the cofactor carbaNAD is still in the region of the initial value even after several days. The results are summarized in Table 2 below:

TABLE 2

| Sample | 0 h | 3 h, 40° C. | 24 h, 40° C. | 45 h, 40° C. |
|---|---|---|---|---|
| Without | 100% | 119% | 145% | 147% |
| cofactor | | | | |
| With NAD | 100% | 105% | 123% | 121% |
| With cNAD | 100% | 106% | 129% | 135% |
| Sample | 0 h | 3 h, 50° C. | 24 h, 50° C. | 45 h, 50° C. |
| Without | 100% | 122% | 90% | 62% |
| cofactor | | | | |
| With NAD | 100% | 99% | 82% | 61% |
| With cNAD | 100% | 109% | 106% | 88% |

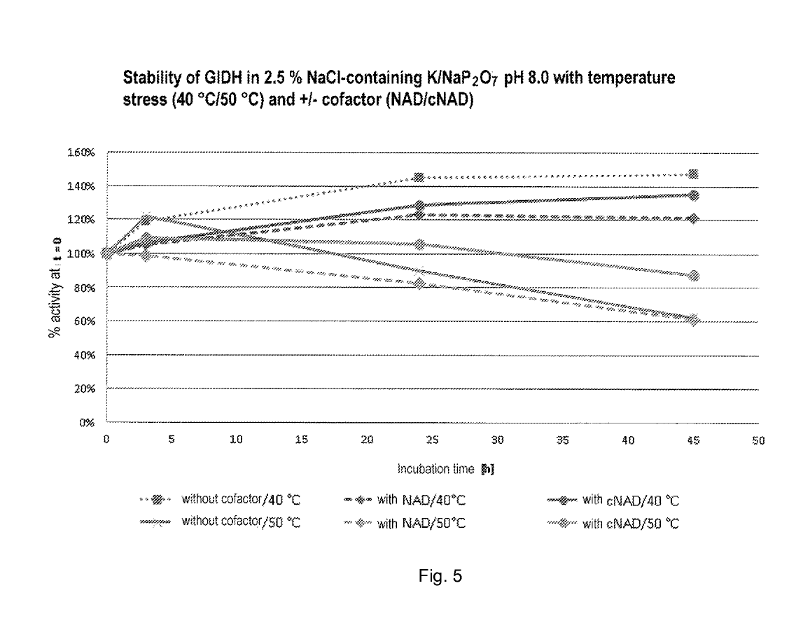

A graphical representation of the results of these determinations is shown in FIG. 5.

All of the patents, patent applications, patent application publications and other publications recited herein are hereby incorporated by reference as if set forth in their entirety.

The present inventive concept has been described in connection with what are presently considered to be the most practical and preferred embodiments. However, the inventive concept has been presented by way of illustration and is not intended to be limited to the disclosed embodiments. Accordingly, one of skill in the art will realize that the inventive concept is intended to encompass all modifications and alternative arrangements within the spirit and scope of the inventive concept as set forth in the appended claims.

The invention claimed is:

1. A method of producing diagnostic test elements, the method comprising the steps of:
(a) providing a batch of an analytical chemical reagent comprising a coenzyme-dependent enzyme and an artificial coenzyme;
(b) coating a first carrier with a first part of the batch of the analytical chemical reagent;
(c) splitting up the coated first carrier into a plurality of first diagnostic test elements;
(d) creating a batch coding for the batch of analytical chemical reagent using at least one of the first diagnostic test elements;
(e) coating a second carrier with a second part of the batch of the analytical chemical reagent;
(f) splitting up the coated second carrier into a plurality of second diagnostic test elements; and
(g) optionally checking and packaging the second diagnostic elements, wherein the second diagnostic elements are each provided with the batch coding created in step (d) before the coated second carrier is split up.

2. The method of claim 1, wherein there is a period of at least about 36 hours between steps (a) and (e) and/or a period of at least about 20 days between steps (e) and (f).

3. The method of claim 1, wherein the batch of the analytical chemical reagent undergoes substantially no change in the chemical and/or physical properties thereof between steps (a) and (e) and/or between steps (e) and (f).

4. The method of claim 1, wherein the batch of analytical chemical reagent is in liquid form, in particular in the form of a suspension.

5. The method of claim 1, wherein the coenzyme-dependent enzyme is selected from the group consisting of a mutated NAD(P)/NAD(P)H-dependent glucose dehydrogenase (EC 1.1.1.47) or a glucose-6-phosphate dehydrogenase (EC 1.1.1.49).

6. A method of claim 1, wherein the artificial coenzyme is selected from the group consisting of an artificial NAD(P)/NAD(P)H compound, carbaNAD, or a compound of formula (I):

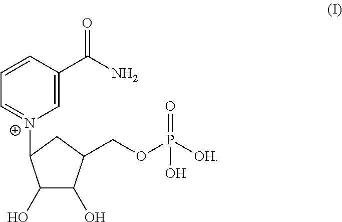 [SMILES: NC(=O)c1ccc[n+](C2CC(COP(=O)(O)O)C(O)C2O)c1]

7. The method of claim 1, where the second carrier is provided with the batch coding before the coating with the second part of the batch of the analytical chemical reagent.

8. The method of claim 1, wherein an optically and/or electronically readable code is used as the batch coding.

9. The method of claim 1, wherein the first diagnostic test elements and/or the second diagnostic test elements each comprise a plurality of test fields.

10. The method of claim 9, wherein each test field is enclosed at least in part by a hydrophobic edge and/or stored in its own substantially closed chamber.

11. The method of claim 10, wherein the hydrophobic edge and/or the chamber walls are formed from a UV-curable material.

12. The method of claim 1, wherein the thickness and/or homogeneity of the layer of the analytical chemical reagent is checked in step (g).

13. The method of claim 1, wherein the method does not comprise checking the water vapour tightness of the packaged diagnostic elements.

14. A diagnostic product, comprising:
(a) at least one diagnostic test element, comprising:
(i) a carrier,
(ii) an analytical chemical reagent comprising a coenzyme-dependent enzyme and an artificial coenzyme, which is applied to the carrier in the form of a layer, and
(iii) a batch coding, and
(b) optionally at least one needle element for scratching the skin.

15. The diagnostic product of claim 14, wherein the diagnostic test element comprises a plurality of test fields.

16. The diagnostic product of claim 15, wherein each test field is enclosed at least in part by a hydrophobic edge and/or stored in its own substantially closed chamber.

17. The diagnostic product of claim 16, wherein the hydrophobic edge and/or the closed chamber are formed from a UV-curable material.

18. The diagnostic product of claim 15, wherein the diagnostic test element is in the form of a test strip, test band, test disc, band magazine or test strip magazine.

19. A method of increasing batch size and/or batch homogeneity, the method comprising the step of:
using an analytical chemical reagent comprising a coenzyme-dependent enzyme and an artificial coenzyme when producing a batch of diagnostic test elements, wherein the analytical chemical reagent increases the batch size and/or batch homogeneity, and wherein individual diagnostic test elements of the batch are substantially identical in each case.

20. The method of claim 19, wherein the batch size is increased by a factor of about 2 when compared to an analytical chemical reagent comprising a coenzyme-dependent enzyme and a native coenzyme.


SEQUENCE LISTING (2 sequences)

SEQ ID NO: 1 - protein, 261 aa, Artificial Sequence
  Mutant of glucose dehydrogenase from Bacillus subtilis
MYPDLKGKVVAITGAASGLGKAMAIRFGKEQAKVVINYYSNKQDPNEVKEEVIKAGGEAVVVQGDVTKEEDVKNIVQTAIKEFGTLDIMINNAGLGNPVPSHEMPLKDWDKVIGTNLTGAFLGSREAIKYFVENDIKGNVINMSSVHEVIPWPLFVHYAASKGGIKLMTKTLALEYAPKGIRVNNIGPGAINTPINAEKFADPKQKADVESMIPMGYIGEPEEIAAVAVWLASKESSYVTGITLFADGGMTKYPSFQAGRG

SEQ ID NO: 2 - protein, 261 aa, Artificial Sequence
  Mutant of glucose dehydrogenase from Bacillus subtilis
MYPDLKGKVVAITGAASGLGKAMAIRFGKEQAKVVINYYSNKQDPNEVKEEVIKAGGEAVVVQGDVTKEEDVKNIVQTAIKEFGTLDIMINNAGLENPVPSHEMPLKDWDKVIGTNLTGAFLGSREAIKYFVENDIKGNVINMSSVHEVIPWPLFVHYAASKGGIKLMTKTLALEYAPKGIRVNNIGPGAINTPINAEKFADPKQKADVESMIPMGYIGEPEEIAAVAVWLASKESSYVTGITLFADGGMTLYPSFQAGRG